(19) United States
(12) Patent Application Publication
(10) Pub. No.: US 2022/0323144 A1
(43) Pub. Date: Oct. 13, 2022

(54) ELECTRODE INSTRUMENT, SURGICAL HANDHELD DEVICE, AND THEIR PRODUCTION METHODS

(71) Applicant: OLYMPUS WINTER & IBE GMBH, Hamburg (DE)

(72) Inventors: Christian BROCKMANN, Hollenstedt (DE); Christoph KNOPF, Stockelsdorf (DE); Jiri LAVICKA, Prerov (CZ); Petr KOMINEK, Prerov (CZ)

(73) Assignee: OLYMPUS WINTER & IBE GMBH, Hamburg (DE)

(21) Appl. No.: 17/719,687

(22) Filed: Apr. 13, 2022

(30) Foreign Application Priority Data
Apr. 13, 2021 (DE) 10 2021 109 175.6

(51) Int. Cl. A61B 18/14

(52) U.S. Cl. CPC A61B 18/1492; A61B 2018/00077

(57) ABSTRACT

An electrode instrument, a surgical handheld device and a production method, by means of which the quality of the aforementioned instruments can be improved. This is achieved by virtue of an electrode instrument for a surgical handheld device having an electrical conductor which is electrically insulated by an insulation in the form of flexible tubing, wherein the conductor is pressed in an electrode carrier, wherein the electrode carrier has a cross section with six pressed sides at the pressed position.

The invention relates to an electrode instrument for surgical handheld device, in particular a resectoscope, as per the preamble of Claim 1. Moreover, the invention relates to a surgical handheld device, in particular a resectoscope, according to Claim 13 and a method for producing a surgical handheld device, in particular a resectoscope, according to Claim 16.

Surgical handheld devices, for example resectoscopes, are used to remove or manipulate body tissue. Typical applications are those in urology. Prostate resection serves as an example in this respect. A radiofrequency tool used in a resectoscope can be an electrode or radiofrequency (RF) electrode which is connected to a radiofrequency generator. The radiofrequency current leads to a plasma forming at the electrode. On account of the interaction of the plasma with the tissue, RF electrodes are particularly well suited to manipulation of the tissue with pinpoint accuracy.

The electrode is detachably attached to a working element of the resectoscope by way of an electrode carrier. Together, electrode and electrode carrier form the so-called electrode instrument. During the treatment of the body tissue, the electrode instrument with the electrode is moved along a longitudinal direction of the resectoscope.

Known electrode instruments are guided in a tube-like shaft, in particular an inner shaft, of the resectoscope, together with an optical unit which may be in the form of a light guide or rod lens system. This shaft extends from the proximal end to the distal end of the resectoscope and for treatment purposes it is guided into the body to be treated, optionally together with an outer shaft. In this case, the optical unit serves to observe a body region during its treatment with the electrode instrument.

As a rule, the electrode instrument consists of the electrode carrier, at the distal end of which the electrode is fastened. In the electrode carrier, an electrical conductor is guided from a proximal end of the electrode carrier to the electrode in order to facilitate the necessary power supply for the electrode. The electrical conductor is electrically insulated from a surrounding electrode casing tube by way of an insulation. In this case, pressings or else crimps, that is to say changes or reductions in cross section, are applied to the electrode instrument in the region of the electrode carrier at one or more positions in order to mechanically keep the components of the electrode carrier together and fasten these to one another.

In this case, it was found to be disadvantageous that a plurality of pressings are applied to the electrode carriers using different tools and this fastening principle requires the exact position of the pressings to be secured or monitored in order to avoid the occurrence of short circuits. Moreover, short circuits may also occur if sharp burrs or edges are unintentionally produced during pressing. This should be avoided. This is because the occurrence of short circuits and the presence of sharp edges or burrs can endanger the patient to be treated. Moreover, the possibility of short circuits occurring reduces the required reliability of the electrode instrument or of the surgical handheld device.

A further disadvantage of the known systems consists of the fact that the components of the electrode carrier are only mechanically stabilized by conventional pressing. However, it is also necessary to seal the interior of the electrode carrier off from moisture and liquids. This is because moisture or liquid, for instance rinsing fluid, entering during a medical treatment can lead to electrical short circuits or creepage currents, and hence endanger the patient and reduce the reliability of the treatment. Further, the ingress of humidity and liquids makes it more difficult to clean and sterilize an electrode carrier, which is required for a medical application. Sealing the interior of the electrode carrier against moisture and liquids is attained by an additional step in the production process, in which a sealant, for example silicone, is applied.

Moreover, work steps are required to setup the production plant so that all components are pressed and sealed exactly. Further steps in the production process consist of checking the quality of the obtained pressings and the seal. The quality control should ensure that all pressed components, from a structural point of view, fit together well, are sealed well and are fully functional. The required additional work steps of sealing and quality control make the production process complicated, labour-intensive and time-consuming, and hence costly.

The invention is based on the object of developing an electrode instrument, a surgical handheld device, in particular a resectoscope, and a production method which solve the aforementioned problems.

One way of achieving this object is described by the features of Claim 1. Accordingly, provision is made for an electrode instrument for a surgical handheld device, in particular a resectoscope, to comprise at least one tube-like electrode carrier, at the distal end of which an electrode is fastened, wherein an electrical conductor is guided within the electrode carrier from a proximal end of the electrode carrier to the electrode and the electrical conductor is electrically insulated from an electrode casing tube by insulation in the form of flexible tubing, wherein the electrical conductor is pressed at at least one position in the electrode carrier, in particular in the electrode casing tube, wherein the electrode carrier, in particular the electrode casing tube, has a cross section with six pressed sides at the pressed position.

High pressure can be exerted on all components within the electrode carrier as a result of this cross-sectional shape of the electrode carrier, in particular of the electrode casing tube. This causes the components of the electrode carrier to be held together and fastened to one another in mechanically secure fashion. At the same time, this cross-sectional shape ensures that the interior of the electrode carrier is sealed against and protected from moisture and liquids. Additional sealing with a sealant is not required. Then again, the pressure exerted in this case is not so high that there may be an unintended deformation of the components of the electrode carrier or an impairment of the function of the electrode carrier or its components.

Additionally, this cross-sectional shape leads to no sharp burrs or edges being produced on the outer and/or inner surface of the electrode carrier, in particular of the electrode casing tube. This precludes the risk of injury to the user, damage to gloves of the user and/or the risk of short circuits. In particular, the present invention can provide for the corners of the cross section to be rounded.

Preferably, the invention provides for the six sides to be of equal length and/or for circular segments of the non-pressed electrode casing tube or rounded or sharp corners of the non-pressed electrode casing tube to be located between the pressed sides such that the cross section is in the form of a hexagonal cross section. In particular, the present invention can provide for even the insulation in the form of flexible tubing to have six sides or a hexagonal cross section at the pressed position. This advantageously contributes to the components of the electrode carrier being securely held together in mechanical fashion and at the same time sealing the interior of the electrode carrier against moisture and liquids. As a result of the pressing, the outer side of the insulation in the form of flexible tubing, in particular, is reshaped into a hexagonal form. The inner side of the insulation in the form of flexible tubing maintains its circular cross section. Further, the present invention can provide for the at least one pressed position to be situated at the distal end of the electrode carrier.

In particular, the present invention can provide for the electrode carrier or each electrode casing tube to have one to five pressed positions, preferably one to three pressed positions, particularly preferably one to two pressed positions along its entire length, wherein each electrode casing tube (18, 42) has the same number or a different number of pressed positions. The presence of a plurality of pressed position leads to an additional mechanical stabilization of the electrode carrier and additional sealing thereof against moisture and liquids. Preferably, provision is made for each electrode casing to have three pressed positions, which serve the transmission of mechanical tensile forces, in a central part. Moreover, it is conceivable for a pressing to be arranged at the distal end of the electrode casing tube. This pressing acts in sealing fashion and prevents water or saline from entering therein. Further, a further pressing can be provided at a proximal end of at least one electrode casing tube, in particular an active side. The at least one pressing at the proximal end serves for sealing purposes and/or for fastening a contact.

Further, it is conceivable that a ratio of an external distance of opposite corners of an electrode casing tube to an external distance of the sides of the electrode casing tube is 1:0.8 to 1:0.95, preferably 1:0.85 to 1:0.9, in particular 1:0.866. A ratio of 0.866 as the theoretical value of a perfect regular hexagon. In reality, the ratio will tend to be slightly larger since the corners are slightly rounded and hence the external distance thereof is less than theory predicts.

In particular, the present invention can provide for a ratio of an internal distance of opposite corners of the electrode casing tube which has been pressed into a hexagonal cross section to an external distance of opposite corners of the pressed insulation to be 1:0.9-1:1, preferably 1:0.99-1:0.999.

In particular, the present invention can further provide for a ratio of an internal diameter of the insulation to a diameter of the electrical conductor at a pressed position to be 1:0.9-1:1, preferably 1:0.99-1:0.999, and/or for a ratio of an external diameter of an electrode casing tube to the external distance of the opposite corners of the electrode casing tube which has been pressed into a hexagonal cross section to be 1:0.9-1:1, preferably 1:0.99-1:0.999. The inner side of the insulation maintains its circular cross section, even after pressing. The specification of an internal diameter of the insulation relates to this inner circular cross section.

The ratios of the dimensions of the components of the electrode carrier to one another within the specified ranges cause the components to fit particularly exactly to one another. This advantageously contributes to securely holding the components of the electrode carrier together in mechanical fashion and, at the same time, sealing the interior of the electrode carrier against moisture and liquids.

Further, the present invention can provide for the internal distance of the opposite corners of the electrode casing tube which has been pressed into a hexagonal cross section to be 0.08 mm-2.8 mm, preferably 0.3 mm-1.8 mm, particularly preferably 0.8 mm-1.3 mm. The invention can further provide for the external distance of the opposite corners of the insulation which has been pressed into a hexagonal cross section to be 0.08 mm-2.8 mm, preferably 0.3 mm-1.8 mm, particularly preferably 0.8 mm-1.3 mm.

The invention can further provide for the diameter of the electrical conductor at the pressed position to be 0.05 mm-1.5 mm, preferably 0.2 mm-1.0 mm, particularly preferably 0.3 mm-0.7 mm. Moreover, according to the invention, provision can be made for the internal diameter of the insulation at the pressed position to be 0.05 mm-1.5 mm, preferably 0.2 mm-1.0 mm, particular preferably 0.3 mm-0.7 mm.

Preferably, the invention can provide for the minimum thickness of the insulation at a pressed position to be 0.02 mm-0.7 mm, preferably 0.1 mm-0.5 mm, particularly preferably 0.2 mm-0.4 mm. Here, the thickness of the insulation at a pressed position is measured as the shortest path between a corner of the insulation which has been pressed into a hexagonal cross section and its inner circular cross section. The thickness of the insulation should not be less than the specified ranges in order to ensure sufficient electrical insulation of the electrical conductor and in order to avoid the presence of creepage currents and electrical short circuits.

Additionally, it is conceivable according to the invention that an external distance of the opposite corners of the electrode casing tube which has been pressed into a hexagonal cross section is 0.1 mm-3.0 mm, preferably 0.5 mm-2.0 mm, particularly preferably 1.0 mm-1.5 mm.

Further, it is conceivable according to the invention that a wall thickness of the electrode casing tube at a pressed position is 0.01 mm-0.6 mm, preferably 0.05 mm-0.4 mm, particularly preferably 0.1 mm-0.3 mm.

The invention can further provide for an internal distance of opposite walls of the electrode casing tube at a pressed position to be 0.08 mm-2.5 mm, preferably 0.4 mm-1.7 mm, particularly preferably 0.8 mm-1.3 mm.

Further, it is conceivable that a length of the pressed position, in particular of a pressing, is 2 mm to 20 mm, preferably 3 mm to 11 mm. It is also conceivable that a distance between two pressed positions, in particular between two pressings, is 2 mm to 10 mm, preferably 5 mm.

The dimensions of the components of the electrode carrier in the specified ranges have the advantage that the components fit particularly exactly to one another. This contributes to securely holding the components of the electrode carrier together in mechanical fashion and, at the same time, sealing the interior of the electrode carrier against moisture and liquids. Further, components with these dimensions yield an electrode carrier which is well suited to the installation in a surgical handheld device, in particular a resectoscope, and which can carry out its functions there to the full.

So that the components of the electrode carriers are securely held together in mechanical fashion and sealed against moisture and liquids at a pressed position, it is also important for the measurements of the components and their dimensions with respect to one another to be of suitable orders of magnitude before pressing or in non-pressed regions or at a non-pressed position. Before pressing or in non-pressed regions, the electrode carrier and all its components preferably have a circular cross section.

Accordingly, provision can be made for a ratio of an internal diameter of the electrode casing tube to an external diameter of the insulation at a non-pressed position to be 1:0.8-1:0.99, preferably 1:0.9-1:0.99. In particular, the internal diameter of the electrode casing tube and the external diameter of the insulation are dimensioned such that the insulation can still be pulled through the tube.

Further, provision can be made for a ratio of an internal diameter of the insulation to a diameter of the electrical conductor at a non-pressed position to be 1:0.8-1:0.99, preferably 1:0.9-1:0.99. In particular, the internal diameter of insulation and the diameter of the conductor are dimensioned such that the insulation can still be pulled over the conductor.

Further, provision can be made for the size of the internal diameter of the electrode tube casing at a non-pressed position to be of the same order as the internal distance of the opposite corners of the electrode casing tube which has been pressed into a hexagonal cross section. The external diameter of the insulation at a non-pressed position can be of the same order as the external distance of the opposite corners of the insulation which has been pressed into a hexagonal cross section. The internal diameter of the insulation at a non-pressed position can be of the same order as the internal diameter of the insulation at a pressed position. The diameter of the electrical conductor at a non-pressed position can be of the same order as the diameter of the electrical conductor at a pressed position. The thickness of the insulation reduces during pressing. The volume between unchanging conductor and reducing outer tube reduces as a result of pressing. As a result of the insulation in the form of flexible tubing already completely filling this volume in a non-pressed state, the flexible tubing is compressed. This ensures that the gap between conductor and tubing and tubing and outer tube is closed. As a result of the combination of plastic deformation of the tube and elastic deformation of the tubing, a permanent pressure of the tubing on the tube and conductor remains in the radial direction. This contact pressure between the components firstly ensures high friction, which can withstand the axial tensile forces, and secondly increased tightness. An external diameter of the electrode casing tube at a non-pressed position can be of the same order as the external distance of opposite corners of the electrode casing tube which has been pressed to form a hexagonal cross section. A wall thickness of the electrode casing tube at a non-pressed position can be of the same order as the wall thickness of the electrode casing tube at a pressed position. Here, the dimensions of the components at a pressed position can be slightly smaller in each case than the dimensions of the corresponding components at a non-pressed position or before pressing.

Further, provision can be made for the electrode instrument according to the invention to comprise one to two electrode carriers, preferably two electrode carriers.

Preferably, the invention can provide for the electrode to be a radiofrequency (RF) electrode. Depending on the application, the electrode, in particular the RF electrode, can be embodied as a cutting loop or as a button electrode, but also the needle, roller, band, etc.

The invention can provide for the electrode casing tube to be manufactured from a metallic material, preferably stainless steel. Further, provision can be made for the insulation to be made from a non-electrically conductive material, preferably Teflon. Moreover, provision can be made for the electrical conductor to be manufactured from a metallic material, preferably stainless steel or copper.

A surgical handheld device, in particular a resectoscope, according to Claim 15 describes a further solution to the problem set forth at the outset. This surgical handheld device, in particular the resectoscope, comprises an electrode instrument according to Claims 1-14. In this case, the electrode instrument comprises at least one tube-like electrode carrier, at the distal end of which an electrode is fastened, wherein an electrical conductor is guided within the electrode carrier from a proximal end of the electrode carrier to the electrode and the electrical conductor is electrically insulated from an electrode casing tube by insulation in the form of flexible tubing, wherein the electrical conductor is pressed at at least one position in the electrode carrier, in particular in the electrode casing tube, wherein the electrode carrier, in particular the electrode casing tube, has a cross section with six pressed sides at the pressed position. The surgical handheld device according to the invention, in particular the resectoscope, preferably comprises an electrode instrument which further has one or more features of the above-described electrode instrument according to the invention. Hence, the surgical handheld device according to the invention, in particular the resectoscope, also has the same advantageous properties as the above-described electrode instrument according to the invention.

A method for producing a surgical handheld device, in particular a resectoscope, for solving the problem set forth at the outset is described by the measures of Claim 16. Claim 16 relates to a method for producing a surgical handheld device, in particular a resectoscope, comprising an electrode instrument with at least one tube-like electrode carrier, at the distal end of which an electrode is fastened, wherein an electrical conductor is guided within the electrode carrier from a proximal end of the electrode carrier to the electrode and the electrical conductor is electrically insulated from an electrode casing tube by insulation in the form of flexible tubing, wherein the electrical conductor is pressed at at least one position in the electrode carrier, in particular in the electrode casing tube, wherein the electrode carrier, in particular the electrode casing tube, adopts a cross section with six pressed sides at the position. The surgical handheld device, in particular the resectoscope, in this case preferably has an electrode instrument which further has one or more features of the above-described electrode instrument according to the invention.

Preferably, the method according to the invention comprises the steps of:

a) providing the at least one electrode carrier;
a1) pulling the insulation tube onto the conductor;
a2) introducing the conductor with insulation tube into the casing tube;
   b) providing a pressing apparatus;
   c) bringing the pressing apparatus into contact with a position of the electrode carrier; and
   d) pressing the electrical conductor in the electrode carrier, in particular in the electrode casing tube, at the position with the aid of the pressing apparatus, wherein the electrode carrier, in particular the electrode casing tube, adopts a hexagonal cross section.

Pressing the electrical conductor in the electrode carrier, in particular in the electrode casing tube and/or the insulation in the form of flexible tubing, to form a cross section with six pressed sides exerts a high pressure on all components within the electrode carrier. As a result, the components of the electrode carrier are held together and fastened to one another in mechanically secure fashion. At the same time, the pressing into this cross-sectional shape advantageously ensures that the interior of the electrode carrier is sealed against and protected from moisture and liquids. However, the pressure exerted in the process is not so high that an unintended deformation of the components of the electrode carrier or an impairment of the function of the electrode carrier or its components may occur.

Additional sealing with a sealant is not required on account of the pressing. Hence, the pressing to form the described cross-sectional shape allows the components of the electrode carrier for the surgical handheld device to be mechanically held together or stabilized and, at the same time, be insulated against the ingress of moisture or liquids. On account of the method according to the invention, only one work step is required to this end, whereas conventional production methods require at least two separate work steps to this end. The number of required steps in quality control is also significantly reduced on account of the method according to the invention. As a result, the production method according to the invention is significantly simplified; work steps are saved. Consequently, the production method according to the invention is also accelerated and saves costs in relation to conventional methods, which is advantageous.

Additionally, pressing into this cross-sectional shape leads to no sharp burrs or edges being generated on the outer surface of the electrode carrier, in particular the electrode casing tube. This avoids the occurrence of short circuits. A further advantage of arranging the pressed cross section within the envelopes of the non-pressed cross section is that this ensures continuous guidance and there are no sections that interfere with guidance protruding beyond the diameter of the casing tube.

The method according to the invention can further provide for the pressing apparatus to be a manual pressing tool or an automated pressing apparatus. Preferably, this can be a pressing apparatus, in particular an automatic pressing apparatus, for producing a plurality of pressings in one operation. In particular, provision can be made for this to be a pressing apparatus, preferably a press, a pressing ram or pliers for producing pressed connections (crimping tool), particularly preferably pliers of which the jaws have a hexagonal cut-out.

A preferred exemplary embodiment of the invention is described below on the basis of the drawing, in which:

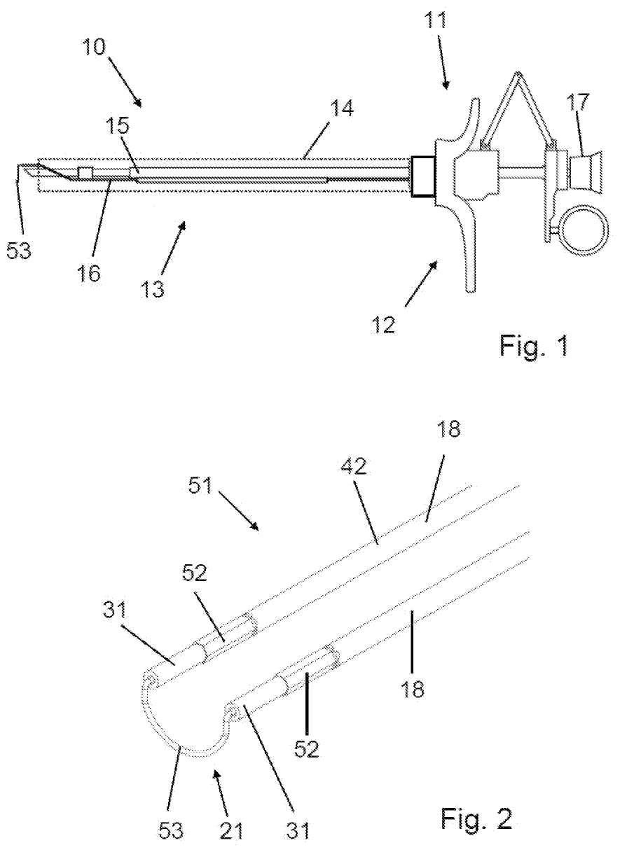

FIG. 1 shows a schematic illustration of a resectoscope,

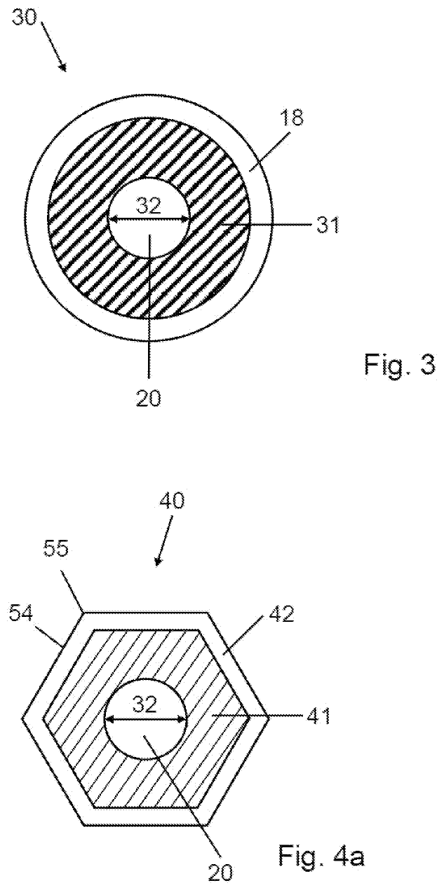

FIG. 2 shows a perspective view of a distal end of an electrode instrument,

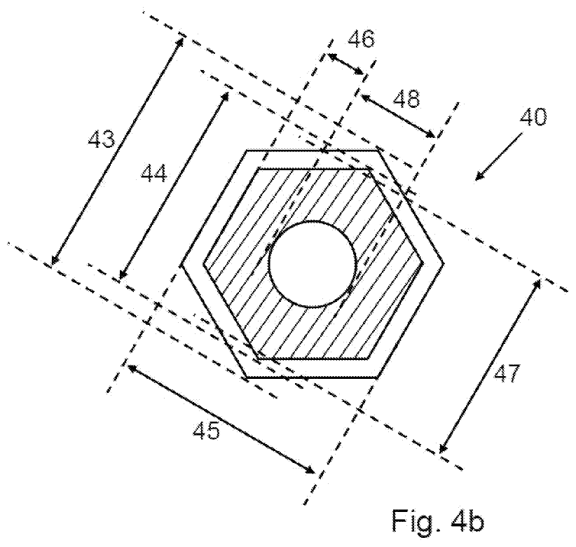

FIG. 3 shows a schematic illustration of a cross section of an electrode carrier at a non-pressed position or prior to pressing,

FIG. 4a shows a schematic illustration of a cross section of an electrode carrier at a pressed position, and

FIG. 4b shows a schematic illustration of a cross section of an electrode carrier as per FIG. 4a.

FIG. 1 illustrates a resectoscope 10 as an example of a surgical handheld device. The resectoscope 10 substantially consists of a working element 11, a handle unit 12, and a tube-like shaft 13. In the exemplary embodiment of a surgical handheld device or resectoscope 10 illustrated here, the tube-like shaft 13 is composed of an outer shaft 14, an inner tube 15, which houses an optical unit, and an electrode instrument 16. An eyepiece 17 is available for a user at the proximal end in order by way of the optical unit to observe the region to be treated medically in front of the distal end 21 of the handheld device.

The electrode instrument 16 extends along the inner tube 15 from the distal end 21 of the resectoscope 10 to the working element 11 at the proximal end. A portion of the electrode instrument 16 has an electrode casing tube 18, 42. The electrode instrument 16 illustrated in FIG. 2 only represents one possible exemplary embodiment. Express reference is made to the fact that the invention described here is not intended to be restricted to the form illustrated here. Rather, it is conceivable that the described invention is also usable in conjunction with differently shaped electrode instruments.

FIG. 2 shows the distal end 21 of the electrode instrument 16. The latter comprises the electrode 53. Electrical energy is able to be applied to this electrode 53, or cutting electrode, by means of an RF generator (not illustrated) and serves to manipulate tissue. Plasma forms around the electrode 53 as a result of applying an RF voltage to the electrode 53. The tissue of a patient can be manipulated or cut by an axial forward and backward movement of the electrode instrument 16.

In addition to the mechanical connection, an electrode carrier 51, which comprises two electrode casing tubes 18, 42 and an electrical conductor 20, also serves to electrically contact the electrode 53. Provision can be made for both the electrode carrier 51 or the electrode casing tube 18, 42 and the electrical conductor 20 to serve as electrical lines or contacts within the electrode carrier 51. Here, the conductor 20 is guided through the electrode casing tubes 18, 42 in its longitudinal direction.

According to the exemplary embodiment illustrated in FIG. 2, the electrode 53, by way of its ends, is mechanically fastened or able to be mechanically fastened to two electrode carriers 51 or to two electrode casing tubes 18, 42. Together with the electrode carriers 51, the electrode 53 represents the essential constituent parts of the electrode instrument 16. Pressings have been applied to the positions 52, as a result of which the electrode carriers 51 have a hexagonal cross section 40 according to the invention at these positions 52. In addition to the parallel guidance, illustrated here, of the electrode carriers 51 or the electrode casing tubes 18, 42, it is also conceivable for the electrode carrier 51 to have a fork-like design and converge in the direction of the proximal end to form a shaft. In this case, the electrode 53 is illustrated as a cutting loop. Additionally, other electrode forms are also conceivable.

FIG. 3 shows a schematic illustration of a cross section 30 of the electrode carrier 51 at a position where the electrode carrier 51 has not been pressed or at a non-pressed position or in a state before pressings are applied. At a non-pressed position or in the state before the pressing, the electrode carrier 51, the electrode casing tubes 18, 42, insulation 31 in the form of flexible tubing and the electrical conductor 20 have a circular cross section. The conductor 20 has a diameter 32.

FIGS. 4a and 4b likewise illustrate a cross section 40 of the electrode carrier 51, but now at a position at which the electrode carrier 51 was pressed according to the invention. As a result of the pressing according to the invention, the electrode carrier 51 has been provided with the hexagonal cross section 40, likewise according to the invention, at this position. Consequently, the electrode casing tubes 18, 42 are provided with six sides 54 as a result of the pressing. These sides 54 can have the same or different lengths and/or can be aligned in pair-wise parallel fashion. Two of the sides 54 in each case form a corner 55, which sides can be a circular segment of the electrode casing tube 18, 42 or can be formed in rounded or angular fashion. After pressing, the electrode casing tubes 18, 42 and the outside of the insulation 41 in the form of flexible tubing have the hexagonal cross section 40 in this case. The electrical conductor 20 and the inner side of the insulation 41 in the form of flexible tubing maintain their circular cross section.

FIG. 4b defines a number of dimensions of the electrode carrier 51 which has been pressed into the hexagonal cross section 40, the dimensions being of importance to the advantageous properties of the electrode carrier. The external distance 43 of the opposite corners 55 of the electrode casing tube 18, 42 that has been pressed into a hexagonal cross section 40 is measured between the outer sides 54 of the opposite corners 55. The internal distance 44 of the opposite corners 55 of the electrode casing tube 18, 42 that has been pressed into the hexagonal cross section 40, by contrast, is measured between the inner sides of the opposite corners 55. The external distance 45 of the opposite walls of the electrode casing tube 18, 42 at a pressed position is determined between the outer sides of the opposite walls. The shortest distance between a side 54 and the inner circular cross section of the insulation 41 is measured as the minimum thickness 46 of the insulation 41 at a pressed position. Specification of the internal diameter 48 of the insulation 41 at a pressed position relates to this inner circular cross section. The external distance 47 of the opposite corners of the insulation 41 that has been pressed to form a hexagonal cross section is ascertained between these opposite corners.

### LIST OF REFERENCE SIGNS

10 Resectoscope
11 Working element
12 Handle unit
13 Shaft
14 Outer shaft
15 Inner tube
16 Electrode instrument
17 Eyepiece
18 Electrode casing tube
20 Electrical conductor
21 Distal end
30 Cross section
31 Insulation
32 Diameter
40 Hexagonal cross section
41 Insulation
42 Electrode casing tube
43 External distance
44 Internal distance
45 External distance
46 Thickness of the insulation
47 External distance
48 Internal diameter of insulation
51 Electrode carrier
52 Pressed position
53 Electrode
54 Side
55 Corner

1. An electrode instrument for a surgical handheld device comprising at least one tube-like electrode carrier, at the distal end of which an electrode is fastened, wherein an electrical conductor is guided within the electrode carrier from a proximal end of the electrode carrier to the electrode and the electrical conductor is electrically insulated from an electrode casing tube by insulation in the form of flexible tubing, wherein the electrical conductor is pressed at at least one position in the electrode carrier, wherein the electrode carrier has a cross section with six pressed sides at the pressed position.

2. The electrode instrument according to claim 1, wherein the six sides are of equal length and/or circular segments of the non-pressed electrode casing tube or rounded or sharp corners of the non-pressed electrode casing tube are located between the pressed sides such that the cross section is in the form of a hexagonal cross section.

3. The electrode instrument according to claim 1, wherein the electrode carrier or each electrode casing tube has one to five pressed positions along its entire length, wherein each electrode casing tube has the same number or a different number of pressed positions.

4. The electrode instrument according to claim 1, wherein a ratio of an external distance of opposite corners of an electrode casing tube to an external distance of the sides of the electrode casing tube is 1:0.8 to 1:0.95.

5. The electrode instrument according to claim 1, wherein a ratio of an internal distance of opposite corners of the electrode casing tube which has been pressed into a hexagonal cross section to an external distance of opposite corners of the pressed insulation is 1:0.9-1:1.

6. The electrode instrument according to claim 1, wherein a ratio of an internal diameter of the insulation to a diameter of the electrical conductor at a pressed position is 1:0.9-1:1.

7. The electrode instrument according to claim 1, wherein a ratio of an external diameter of an electrode casing tube to the external distance of the opposite corners of the electrode casing tube which has been pressed into a hexagonal cross section is 1:0.9-1:1.

8. The electrode instrument according to claim 1, wherein the thickness of the insulation at a pressed position is 0.02 mm-0.7 mm.

9. The electrode instrument according to claim 1, wherein the external distance of the opposite corners of the electrode casing tube which has been pressed into a hexagonal cross section is 0.1 mm-3.0 mm.

10. The electrode instrument according to claim 1, wherein the diameter of the electrical conductor at a pressed position is 0.05 mm-1.5 mm.

11. The electrode instrument according to claim 1, wherein an external distance of the opposite walls of the electrode casing tube at a pressed position is 0.08 mm-2.5 mm.

12. The electrode instrument according to claim 1, wherein a length of the pressed position is 2 mm to 20 mm.

13. The electrode instrument according to claim 1, wherein a distance between two pressed positions is 2 mm to 10 mm.

14. The electrode instrument according to claim 1, wherein at least one pressed position is at the distal end of the electrode carrier.

15. A surgical handheld device comprising an electrode instrument according to claim 1, wherein with at least one tube-like electrode carrier, at the distal end of which an electrode is fastened, wherein an electrical conductor is guided within the electrode carrier from a proximal end of the electrode carrier to the electrode and the electrical conductor is electrically insulated from at least one electrode casing tube by insulation in the form of flexible tubing, wherein the electrical conductor is pressed at at least one position in the electrode carrier wherein the electrode carrier has a cross section with six pressed sides at the pressed position.

16. A method for producing a surgical handheld device comprising an electrode instrument according to claim 1, wherein at least one tube-like electrode carrier, at the distal end of which an electrode is fastened, wherein an electrical conductor is guided within the electrode carrier from a proximal end of the electrode carrier to the electrode and the electrical conductor is electrically insulated from an electrode casing tube by insulation in the form of flexible tubing, wherein the electrical conductor is pressed at at least one position in the electrode carrier wherein the electrode carrier adopts a cross section with six pressed sides at the position.

17. The method for producing a surgical handheld device according to claim 16, wherein the method comprises the steps of:
a) providing the at least one electrode carrier;
a1) pulling the insulation tube onto the conductor;
a2) introducing the conductor with insulation tube into the casing tube;
b) providing a pressing apparatus;
c) bringing the pressing apparatus into contact with a position of the electrode carrier; and
d) pressing the electrical conductor in the electrode carrier at the position with the aid of the pressing apparatus, wherein the electrode carrier adopts a cross section with six pressed sides.
